(19) United States
(12) Patent Application Publication
(10) Pub. No.: US 2007/0282177 A1
(43) Pub. Date: Dec. 6, 2007

(54) Arrangement of equipment for remote monitoring of bodily functions

(75) Inventor: Ulrich Pilz, Berlin (DE)

Correspondence Address: VENABLE LLP, P.O. BOX 34385, WASHINGTON, DC, 20043-9998, US

(73) Assignee: UbiCom Gesellschaft fur Telekommunikation mbH, Hohen Neuendorf (DE)

(21) Appl. No.: 10/419,965

(22) Filed: Apr. 22, 2003

(30) Foreign Application Priority Data
Apr. 22, 2002 (DE) 02 008 958.7

(51) Int. Cl. A61B 5/00; G08B 23/00

(52) U.S. Cl. 600/301; 340/573.1

(57) ABSTRACT

An arrangement of equipment for the remote monitoring of bodily functions of a mammal under medical care, comprises: a function-measurement device to measure a physiological quantity relevant to bodily function; a first display unit disposed remotely from the function-measurement device and monitored by a responsible medical person, to display the measured values of the measured physiological quantity and a function characterization derived therefrom; an input unit disposed remotely from the function-measurement device, for the input of control commands for the function-measurement device, and to input warning signals; and a bi-directional public telecommunication-network connection with an outward channel between the function-measurement device and the first display unit and a return channel between the input unit and the function-measurement device, where, for each connected device, there is provided an interface adapted to a transmission protocol of the telecommunication network.

### CROSS-REFERENCE TO RELATED APPLICATIONS

The present application claims the benefit of European Priority Application No. 02 008 958.7 filed in Europe on Apr. 22, 2002, the disclosure of which is incorporated herein by reference.

### BACKGROUND OF THE INVENTION

1. Field of the Invention

The present invention relates to monitoring physiologic data in real time, and more specifically to monitoring such data remotely.

2. Related Art

Advances in medical technology have made it possible to treat a large number of diseases and disorders in such a way that although the affected people are not ultimately cured, they can have a good quality of life despite the disease. However, such illnesses (e.g., diabetes, various heart diseases, asthma, diseases of the rheumatic type, cancers, Alzheimer's disease, allergies) demand continuous medical care of the patients, because medications must be taken regularly, and long-term diagnostic monitoring by appropriate measurement devices is required. Furthermore, the diagnostic data obtained from the patient must regularly be checked by a specialist, and the patient must remain under the care of a physician so that the progress of the disorder can be made as nearly optimal for the patient as possible.

Further, advances in medical technology for the treatment of the chronically ill (e.g., installations to allow asthmatics to breathe oxygen) and for the collection of diagnostic data (e.g., glucose measurement in diabetes) have become so well developed that the patient can manage them autonomously while ambulant.

The generally known state of the art in cardiac and circulatory diseases includes heart clinics in which infarct patients spend weeks and months under observation. The care system is supplemented by clinic-based physicians who can prepare ECGs and provide the patients with 24-hour recording devices, so that a daily visit by the doctor can be eliminated. Systems are also known in which a patient can hold an instrument similar to a mobile phone against his chest whenever he thinks it necessary, so as to transmit data about his heart condition to a physician's practice by way of a wireless network.

For asthmatics, there are devices to measure pulmonary function that the patient can operate independently. However, even in combination with visits to a doctor, this is not sufficient for successful therapy, because the latter requires a regular intake of medicines (particularly during symptom-free periods, when many patients tend to neglect their medication) as well as adjustment of the therapy schedule by the doctor to suit particular situations. In many cases patients with chronic lung disorders also need special breathing aids (to increase oxygen content and filter out dust and pollen), the maintenance of which must also be carried out by specially trained workers.

From the areas of space travel and sports medicine it is also known that the bodily functions of individual people (astronauts in a spaceship, high-performance athletes during running or training) can be monitored by measurement devices carried on the subject's body or in any case disposed near the subject of the investigation, the results of the measurement being transmitted over a specially established wireless route to a central unit, such as a ground station, an accompanying vehicle or a training centre, for evaluation and observation. There the data can be evaluated nearly in real time. In some cases means are also provided for wireless transmission of instructions to the investigated person, for instance regarding how to adjust the level of physical activity, or that certain manipulations should be carried out.

These last solutions to the problem of remote monitoring are unsuitable for a large health-monitoring system from which a large number of patients, especially the chronically ill, can benefit and which in its medical components can be operated by a widely branching network of clinics, physicians' practices and other medical-care sites, because the means of information transmission are very elaborate and because of specific technical properties of the transmission routes employed.

### SUMMARY OF THE INVENTION

The invention includes function-measurement devices situated near or on the subject, i.e. the patient, of the investigation, which are connected by way of a public telecommunication network to display units and/or automatic monitoring and control apparatus situated in the clinical or ambulant region. The use of a public network ensures not only considerable advantages of economy in comparison to specially established wireless routes, but also a practically unlimited system capacity and extraordinarily great availability of the communication link and hence convincing reliability of monitoring. When a mobile wireless network is employed, which is preferable from the present viewpoint, there is the additional advantage that the patient can be reached practically everywhere within range, without any gaps, at least in the industrialized countries.

The invention implements, within the framework of the above-mentioned connection by a telecommunication network, both an outgoing and an incoming channel. The data obtained from the patient by measurement or evaluation are sent to the display unit or monitoring and control apparatus. Instructions for activities can be sent to the patient, or control commands to a measurement or detection device associated with the patient or also to a device provided for remote-controlled therapy.

In a first implementation, the invention incorporates the active participation of medical personnel in the remote monitoring and optional remote-controlled therapy of the patient. In a second implementation of the invention, a substantially automatic evaluation of the measurements obtained from the patient is provided for the automatic generation of control commands or instructions to the patient. Either of the two implementations can be preferred, depending on the specific state of development of a national health system within which the invention is being employed, and in particular on the specific nature of the patient's illness and the therapeutic measures being undertaken.

In larger systems of the kind in accordance with the invention it may well be advantageous to combine the two variants in such a way that, depending on the illness, primacy will be given to treatment either initiated by medical personnel or performed automatically. It should be understood that normally at least every automated patient-care treatment will be documented and subjected to evaluation by a physician together with the data and/or event that initiated the treatment, whether temporally in parallel or subsequently.

In view of the above explanations, it should further be apparent that an established system of the kind in accordance with the invention will comprise many function-measurement devices associated with a large group of patients, and a whole network of display units and monitoring and control apparatus disposed at the medical end, so that a correspondingly large number of interfaces with a telecommunication network or even several telecommunication networks are provided. The incorporation of several independent telecommunication networks into the system may be necessary in particular for system solutions that extend across national boundaries, but for reasons of competition and economy it can also play a role in national systems. The devices connected to the communication network on both sides can of course be quite different from one another—all that is required is to establish a transmission standard (or several compatible standards) for their communication and interaction.

In an embodiment preferred for its practical usefulness, the proposed system comprises in addition to the fundamental components, i.e. the function-measurement devices, display units or monitoring and control apparatus, and telecom network with outward and return channels, therapeutic devices and/or additional display units at the patient's end, so that in response to a monitoring result that demands a medical reaction instructions as to what should be done can immediately be displayed to the patient, or the therapeutic device can be directly controlled so that it automatically initiates suitable therapy.

By application of the invention patients at home or travelling can receive treatment of approximately the same quality as they would under stationary conditions in a clinic. Over the spatial distance it is possible, first, for the medical data specific to the individual to be transmitted and evaluated by a specialist physician. Second, monitoring ensures that medication is being taken as prescribed. Third, there can be a direct, video-supported contact between the patient or the patient's caregivers and the doctor. Fourth, medical devices can be operated and maintained by remote control. Fifth, the patient can use an Internet portal at all times to access personal data, advice from the doctor, servicing dates for the devices being used, and so on.

In carrying out medical studies, but also when particular routines are to be followed, the compliance of the subjects, i.e. the degree of precision with which they conform to the prescriptions for taking medicines and using equipment, is of crucial significance. Here the invention contributes to recording the data reliably, managing the data and testing the data for correctness. The invention also enables devices to be employed in a simple manner for objective measurement of patient compliance.

In an exemplary embodiment, the present invention can be a arrangement of equipment for the remote monitoring of bodily functions of a mammal under medical care, comprising: a function-measurement device to measure a physiological quantity relevant to bodily function; a first display unit disposed remotely from the function-measurement device and monitored by a responsible medical person, to display the measured values of the measured physiological quantity or a function characterization derived therefrom; an input unit disposed remotely from the function-measurement device, for the input of control commands for the function-measurement device, and to input warning signals; and a bi-directional public telecommunication-network connection with an outward channel between the function-measurement device and the first display unit and a return channel between the input unit and the function-measurement device, wherein for each connected device there is provided an interface adapted to a transmission protocol of the telecommunication network.

In another exemplary embodiment, the present invention can be a arrangement of equipment for the remote monitoring of bodily functions of a mammal under medical care comprising: a function-measurement device to measure a physiological quantity relevant to bodily function; an automatic monitoring and control unit disposed remotely from the function-measurement device, to evaluate the measured values of the physiological quantity or a function characterization derived therefrom, and to generate control commands in response to the result of the evaluation for the function-measurement device; and a bi-directional public telecommunication-network connection with an outward channel between the function-measurement device and the automatic monitoring and control unit and a return channel between the automatic monitoring and control unit and the function-measurement device, wherein for the connected device there is provided an interface adapted to a transmission protocol of the telecommunication network.

Further features and advantages of the invention, as well as the structure and operation of various embodiments of the invention, are described in detail below with reference to the accompanying drawings.

### BRIEF DESCRIPTION OF THE DRAWINGS

Further advantages and useful features of the invention will be apparent from the subordinate claims and from the following description of preferred exemplary embodiments with reference to the figures, wherein

FIG. 1 is a sketch showing the principles of an arrangement according to a first embodiment of the invention;

FIG. 2 is a sketch showing the principles of an arrangement according to a second embodiment of the invention;

FIG. 3 is a sketch showing the principles of an arrangement according to a third embodiment of the invention; and

FIG. 4 is a sketch showing the principles of an arrangement according to a fourth embodiment of the invention.

### DETAILED DESCRIPTION OF THE INVENTION

A preferred embodiment of the invention is discussed in detail below wherein like reference numbers generally indicate identical, functionally similar, and/or structurally similar elements. While specific exemplary embodiments are discussed, it should be understood that this is done for illustration purposes only. A person skilled in the relevant art will recognize that other components and configurations can be used without departing from the spirit and scope of the invention.

The invention relates to an arrangement of equipment for the remote monitoring of bodily functions of a human under medical care, or a mammal under the care of a veterinarian. The functions that can be served by equipment thus arranged include monitoring an athlete in the context of sports medicine, or a patient with a chronic disorder, and, where appropriate, monitoring of the bodily functions of highly valuable animals in high-performance animal husbandry.

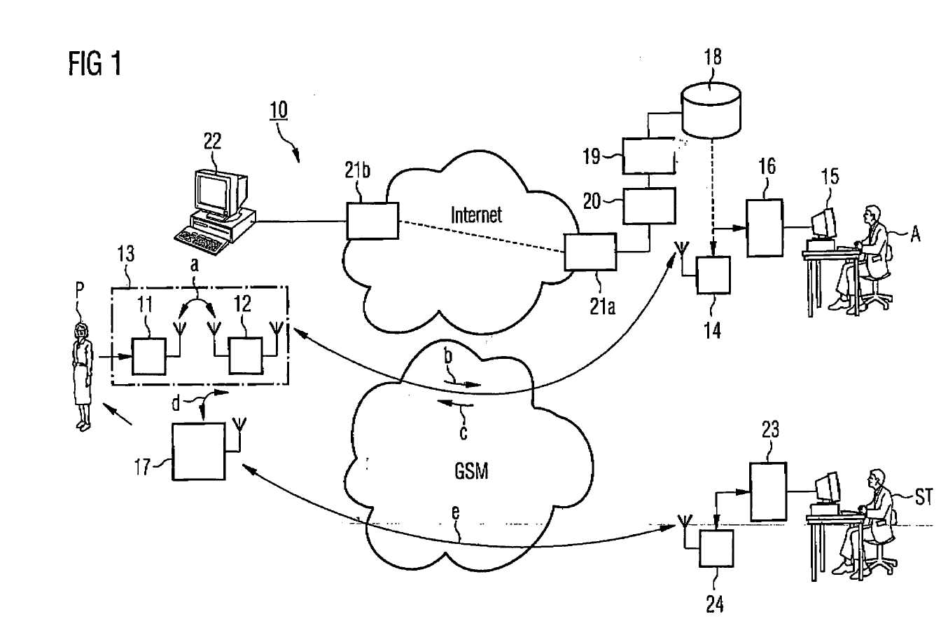

FIG. 1 shows as a first embodiment of the invention an arrangement 10 of equipment for the care of an asthmatic patient P who is some distance away from a centre specializing in pulmonary disorders. The patient P has at his disposal a simply constructed device 11 for the diagnosis of pulmonary function, which he carries on his body and can operate with no problems. The device 11 incorporates a Bluetooth card and is in permanent connection, by way of a short-range wireless connection a according to the Bluetooth standard, with a similarly equipped mobile wireless terminal 12.

The mobile wireless terminal 12 can, for example, be a GSM mobile telephone with a data input as an interface to a mobile wireless network GSM (here shown only schematically), which in case of need can establish a long-range wireless connection b to another mobile wireless terminal 14 near a physician A in the specialized pulmonary centre. The diagnosis device 11 and the mobile wireless terminal 12 on the patient's side together can also be termed the patient-condition monitoring unit 13.

The pulmonary-function diagnosis device 11 stores at least one threshold value for a quantity characterizing the pulmonary function or breathing activity, for instance the respiratory rate and/or the respired volume. The device 11 is designed to detect the corresponding measured values and compare these with the threshold value. If the measured values pass significantly below the threshold value or values, the comparator unit automatically sends an alarm signal over the Bluetooth connection a to the mobile telephone 12, which then likewise automatically initiates the transmission of an alarm announcement over the mobile wireless connection b to the mobile telephone 14 on the pulmonary centre side, causing an alarm announcement to appear on a display unit 15, for example, a PC screen, attached thereto.

Using the associated PC 16 as input unit, the physician A can then give a remote-control command to activate a therapeutic device 17, for example, an aerosol applicator, that is continually in the vicinity of the patient or carried by the patient. The command is transmitted by way of the return channel comprising the physician's mobile telephone 14 and the mobile telephone 12 on the patient's side, plus a second Bluetooth connection between the mobile telephone 12 and the aerosol applicator 17, with the result that a pre-specified therapy consisting of supplying the patient with aerosol is automatically initiated. That is, the therapy is given regardless of the patient's condition and whether or not he is capable of taking action himself, and hence is reliable even in case of a severe, acute asthma attack.

When an irregularity in the patient's breathing is detected, the pulmonary-function data measured and the alarm signal identifying an irregularity are sent, and provided with a time stamp, to the display unit 15 and to a central patient-data memory 18 in the specialized centre, where the information is stored with reference to the individual patient. The data management is achieved by an associated data-management unit 19, with which is associated an authorization testing stage 20 to determine whether external attempts to gain access are authorized. By way of a data-net interface 21a the patient-data memory 18 can be connected to the Internet, and on the patient's side, by way of the mobile telephone 12 or a PC 22 and a data-net interface 21b, the patient can view his own data from the past by entering an appropriate authorization code.

The aerosol applicator 17, by way of its Bluetooth connection c to the patient's mobile telephone 12, receives the appropriate control signals from the medical centre in case therapy is needed, and, at regular servicing intervals, receives function-monitoring signals from a remote service technician ST, who enters them into his PC 23 from which they are transmitted by a mobile wireless terminal 24 on the technician's side and a separate connection e in the mobile wireless network GSM. The PC 23 of the service technician ST thus has the function of a remote monitoring and maintenance device, and the wireless connection in this case likewise ensures that the necessary activities can be carried out regardless of the current location of the patient.

Servicing and repair visits by the service technician ST to the patient P are not rendered fundamentally superfluous by these means of remote-controlled maintenance, but their frequency can be reduced and efficiency increased. Furthermore, it becomes possible for the service technician to contact the patient directly by way of the mobile wireless connection or, alternatively, over the Internet, in order to make an appointment for servicing that must be done on site; however, to preserve the clarity of the illustration these possibilities are not indicated in the figure.

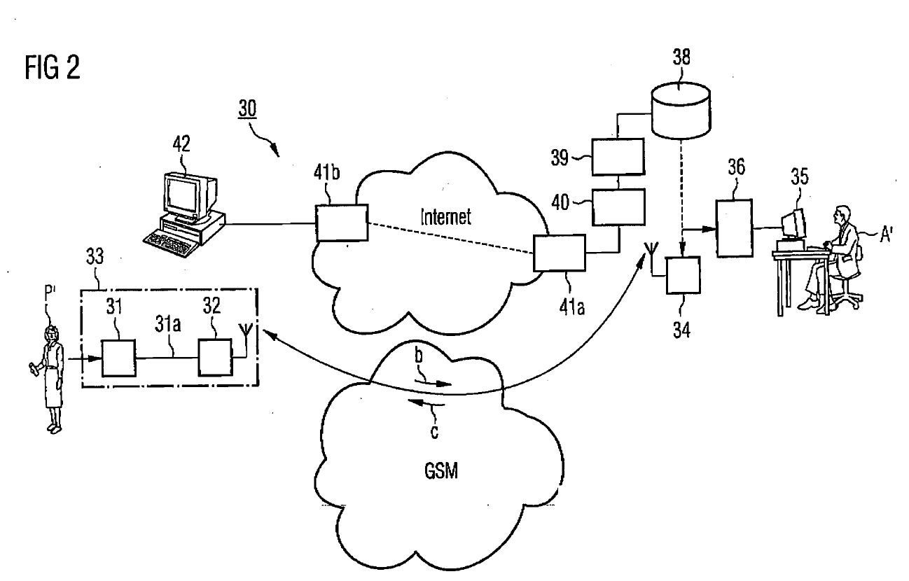

In FIG. 2 another example of a system in accordance with the invention is sketched, in this case an arrangement 30 of equipment with which a patient P′ with high blood pressure can be treated by remote control by a physician A′ in a medical practice. Components having substantially the same function as components in the arrangement according to FIG. 1 are identified by the same reference numerals, plus twenty, as in the FIG. 1, and—insofar as there are no special features to be noted in the present arrangement—are not explained again.

The arrangement 30 comprises a blood-pressure measurement device 31, which is carried and operated by the patient and is connected to the patient's mobile telephone 32 by a data cable 31a. The blood-pressure measurement device 31 and the mobile telephone 32 again (as in the first example) form a patient-monitoring unit 33.

The physician A′ has at his disposal the same system components that were available in the specialized pulmonary centre in the first example, and the transmission to him of signals indicating blood-pressure values that should be considered irregular is the same as in the first example. A difference is that here the patient has no therapeutic device available, but rather when his blood pressure becomes unacceptably high or low, receives from the physician specific advice as to medication by way of the return channel e of the long-range mobile wireless connection between the mobile telephone 34 on the physician's side and the mobile telephone 32 on the patient's side. With this kind of signal transmission, the patient's mobile telephone 32 serves simultaneously as a display unit, by way of which the patient receives instructions about taking medications to adjust blood pressure, in the form of spoken output or a display.

Regarding the availability of a patient data bank, this embodiment is also the same as the first example, in FIG. 1; but the means of remote-controlled maintenance can be eliminated in the diagnosis device employed here, which is produced in large numbers, operates reliably over long periods and does not include a therapeutic device.

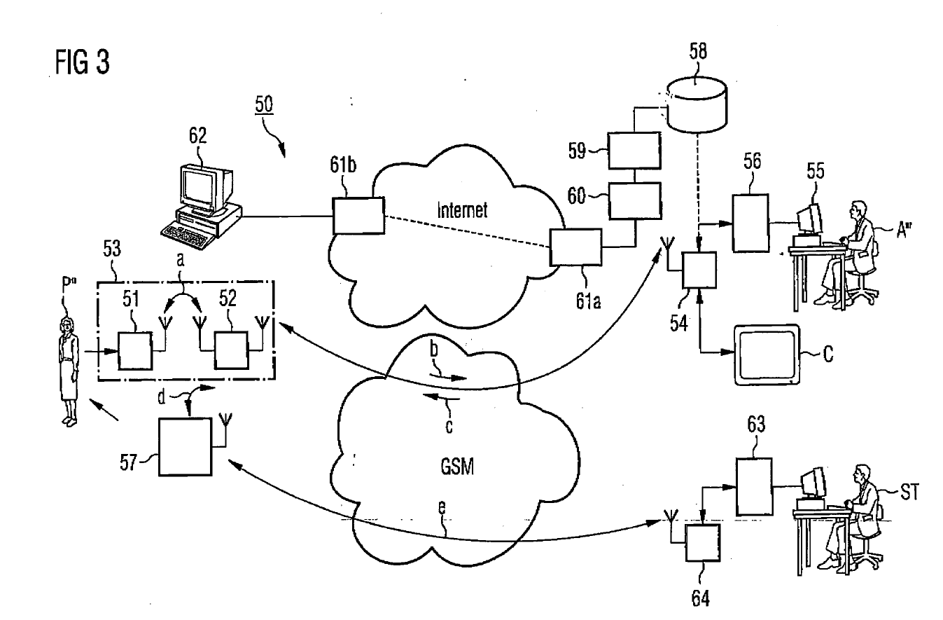

FIG. 3 shows—likewise schematically—a third arrangement 50 of equipment for remote diagnosis and therapy, in this case for a patient suffering from chronic diabetes. It includes a central therapy-control unit C supervised by a diabetes physician A′ in a diabetes centre. The basic construction of this arrangement corresponds to that of the first example, shown in FIG. 1, so that here again components with substantially the same action are identified by the same reference numerals as in FIG. 1, plus forty, and are not described again.

The diagnosis device concerned here is an implanted glucometer 51, and the therapeutic device is a likewise implanted insulin pump 57 under remote control. The outward and return channels of a mobile wireless connection between these devices on the patient's side and the diabetes centre are basically configured as in FIG. 1—except that now in addition to the connection to a PC 56 of the diabetes physician, a direct connection to the therapy-control unit C is also made as soon as the glucometer detects questionable blood-sugar levels.

In response to an evaluation of the transmitted blood-sugar values the therapy-control device C, with no need for intervention by the physician, sends out a control command for the insulin pump. The control command is transmitted through the return channel of the mobile wireless connection to the insulin pump and activates the latter in the pre-specified manner, or alters the amount of insulin injected.

Particular testing and maintenance functions for the glucometer and the insulin pump are implemented as remote-controlled maintenance, analogous to the system according to FIG. 1, and the patient-data storage in a data bank and the data management are also correspondingly organized.

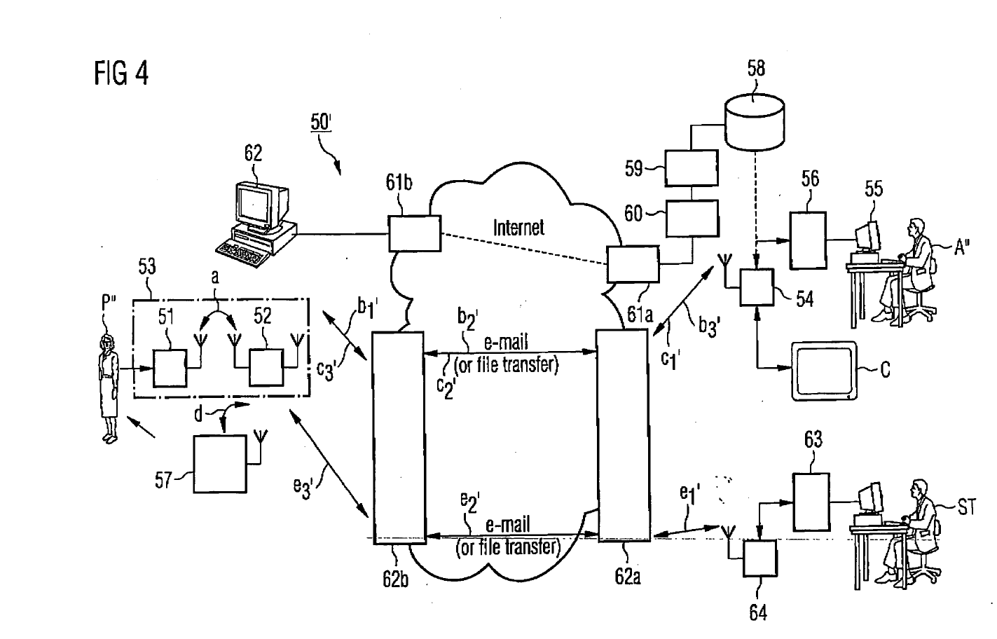

FIG. 4 shows, using the same reference numerals as those used in FIG. 3 for the third embodiment, a fourth embodiment, distinguished from the third with respect to the network structure for the relevant information-transfer events. The arrangement as a whole is identified by the reference numeral 50′ and differs from the third embodiment in that the Internet is used for transmission of all the information.

The long-range information transfer between the patient-condition monitoring unit 53 and the mobile wireless terminal 54 on the clinic's or physician's side as well as in the opposite direction, between the latter and the patient-condition monitoring unit, is in each case subdivided into three sections, b1′ to b3′ and c1′ to c3′, respectively. The first and last sections in each case are formed by a mobile wireless connection between the associated terminal and a gateway 62a or 62b to the internet, whereas the middle section is established by sending an e-mail or a file transfer within the data network. The gateways 62a, 62b carry out an SMS/e-mail or e-mail/SMS conversion in a manner known per se and made available as a service by providers.

In this embodiment the data transfer between the mobile wireless terminal 64 of the service technician ST and the insulin pump 57 (by way of the mobile wireless element 52) is organized in the same way. For the data transfer concerned here it is also possible to provide another combination of generally used transmission formats with corresponding conversion, such as a combination of SMS and fax transmission, or of e-mail and fax transmission.

Specifically for patients with cardiac disorders, the function-measurement device provided is a pulse-rate or heart-rhythm detector, both of which are known in the art. Alternatively or in addition, patients with chronic circulatory diseases can have as function-measurement devices a blood-pressure meter and/or an oximeter to measure the oxygen content of the blood. Additional measurement devices can also be employed within the framework of the proposed system, such as are used in patients with implanted cardiac pacemakers to supplement the data provided by the heart-rate detector with other reliable data—for instance, a sensor to detect body position (standing/reclining) or an accelerometer to detect strenuous body activities.

In an exemplary embodiment of the present invention, diabetics are advantageously given a means for the periodic, automatic measurement of blood sugar (glucose) content. In another exemplary embodiment of the present invention, patients with severe airway diseases have a device to detect pulmonary performance or respiratory rhythm, which is known in the art. In another exemplary embodiment of the present invention, patients with, in some cases potentially lethal, nerve diseases can carry a portable EEG recorder to detect the brain currents. This list is not exhaustive and is meant merely to illustrate important applications of the proposed system and the data-collection devices that can be used therewith.

For the above-mentioned cardiac patients, the therapeutic device preferably takes the form of a cardiac pacemaker, known in the art. In the case of high-frequency disturbances of cardiac rhythm that might lead to fibrillation, the therapeutic device can be a defibrillator. These devices for correcting cardiac rhythm have long been available in designs suitable for implantation and remote control by way of short-range telemetry, and they can and should also be employed in the system proposed here.

Likewise for cardiac patients, but also for the treatment of other diseases that can develop into acute risk conditions requiring urgent medication, medicine-dosing devices can also be employed as therapeutic devices. These too are preferably controllable by remote means and of course are particularly advantageous (in the form of the known implantable insulin pumps) primarily for patients with severe diabetes. A simple and more economical way to implement the dispensing of medicine in conformity with this system consists in instructing patients to take a medicine in the form of pills or drops, or to give themselves an injection. For a permanently reliable treatment with medication, however, since the results are independent of the patients' following such instructions, the more expensive medicine-dosing devices have considerable advantages in terms of security.

For patients with severe airway diseases, again especially if the disease might rapidly become acutely dangerous (as in the case of asthmatics suddenly exposed to allergens), remote-controlled breathing aids can be used in conformity with the system. These can in particular comprise an oxygen or aerosol generator. Here, again, the less costly variant specifically in the case of asthmatics consists of instructing the patients to make use of a device that they operate themselves—but devices that can be centrally controlled by way of the return channel of the telecom connection have security advantages in some of these cases as well.

In addition to the above-mentioned devices, which can be considered diagnostic or therapeutic devices in the narrow sense, in an advantageous embodiment the system additionally comprises a picture-recording device on the patient's side, as well as “centrally” (i.e., at a distance from the picture-recording device) a video reception and display device, so that the pictures taken by the patient can be displayed to the medical personnel to facilitate their evaluation of the patient's condition. In cases of severe chronic dermatological diseases this picture-recording device (video camera) can also replace the function-measurement device, and the picture it delivers is evaluated in analogy to the measured data in the case of other diseases.

The interfaces associated with individual system components to connect them to the telecom network take the form, in a preferred embodiment, of mobile wireless terminals with inputs and outputs suitable for data. The term “mobile wireless terminal” in the context of the invention should be understood to include not only normal mobile telephones but also transmitter/receiver elements with reduced functionality (e.g., with no means for speech transmission), as well as hand-held PCs or personal digital assistants (PDA) with a mobile wireless component.

Outward and return channels for data transmission in SMS or EMS format or based on the wireless application protocol (WAP) standard (specifically per WAP push) can be designed in the same way as is known for other applications. In the outward channel the transmission is preferably activated automatically by way of the function-measurement device or the picture-recording device, whereas in the return channel it is initiated either manually by the medical personnel who are evaluating the results, or automatically by the monitoring and control unit in the patient-care centre. The outward and return channels can also be permanent, packet-oriented exclusive connections according to the GPRS standard within a GSM network or according to the future UMTS standard.

In a preferred embodiment, to preserve the data, on the physician's side there is provided a central data bank to store the relevant transmitted measured data or information identifiers, and associated therewith is in particular a central data-bank management system for the management of these data for the medical care devices. The data-preservation system is preferred because it is accessible on the basis of appropriate authorization. With graded authorization tests and access controls, both the authorized medical personnel (e.g., the house doctor) and also the patient can be granted simple access to the stored data from any desired terminal devices.

The above-mentioned function-measurement devices or therapeutic device can be connected to the system by way of an outward and/or return channel that comprises a wireless short-range connection to an associated interface of the telecom network. The above-mentioned picture-recording device on the patient's side (e.g. a video camera or web-cam) can likewise have a similar wireless connection, for example, a wireless LAN, a Bluetooth or a DECT connection. With respect to cost, the Bluetooth and the DECT connections appear advantageous, and, according to preliminary information, the Bluetooth standard additionally offers advantages in reliability.

The embodiment of the invention is not limited to the aspects emphasized above or to the preferred exemplary embodiments, but is equally possible in a large number of modifications that are within the competence of a person skilled in the art.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

What is claimed is:

1. An arrangement of equipment for the remote monitoring of bodily functions of a mammal under medical care, comprising:
a function-measurement device to measure a physiological quantity relevant to bodily function;
a first display unit disposed remotely from the function-measurement device, for being monitored by a responsible medical person, to display the measured values of at least one of the measured physiological quantity and a function characterization derived therefrom;
an input unit disposed remotely from the function-measurement device for the input of control commands for the function-measurement device; and
a bi-directional public telecommunication-network connection with an outward channel between the function-measurement device and the first display unit and a return channel between the input unit and the function-measurement device, wherein for each connected device there is provided an interface adapted to a transmission protocol of the telecommunication network.

2. The arrangement of equipment according to claim 1, further comprising:
a video camera disposed at the mammal; and
a video receiving and display device disposed remotely from the video camera and connected thereto by the outward channel of the public telecommunication-network connection, to receive and display the pictures of the mammal recorded by the video camera.

3. The arrangement of equipment according to claim 1, further comprising a second display unit disposed at the mammal to display warning signals or information entered from the input unit.

4. The arrangement of equipment according to claim 1, further comprising at least one therapeutic device disposed at the mammal to provide a therapy in response to a control command entered at the input unit.

5. The arrangement of equipment according to claim 1, further comprising:
a wireless short-range connection between the function-measurement device and at least one additional device disposed at the mammal; and
associated with the additional device, an interface of the telecommunication network according to one of the Bluetooth, wireless LAN, and DECT standards.

6. The arrangement of equipment according to claim 5, wherein said additional device disposed at the mammal is one of a video camera, a therapeutic device, and a second display unit.

7. The arrangement of equipment according to claim 1, wherein the function-measurement device is one of a pulse meter to detect the pulse rate, and a cardiac-rhythm detector.

8. The arrangement of equipment according to claim 1, wherein the function-measurement device is a blood pressure meter.

9. The arrangement of equipment according to claim 1, wherein the function-measurement device is an oximeter device to detect the blood oxygen content.

10. The arrangement of equipment according to claim 1, wherein the function-measurement device is a glucose-measurement device to detect the blood-sugar content.

11. The arrangement of equipment according to claim 1, wherein the function-measurement device is a device to detect one of a respiratory rhythm and pulmonary performance.

12. The arrangement of equipment according to claim 1, wherein the function-measurement device is an EEG-recording device to detect the brain currents.

13. The arrangement of equipment according to claim 1, wherein the function-measurement device comprises an evaluation device to obtain a function characterization from the collected data.

14. The arrangement of equipment according to claim 4, wherein the therapeutic device is a remote-controlled, implanted cardiac pacemaker.

15. The arrangement of equipment according to claim 4, wherein the therapeutic device is a remote-controlled, implanted defibrillator.

16. The arrangement of equipment according to claim 4, wherein the therapeutic device is a remote-controlled breathing aid, with an oxygen or aerosol generator.

17. The arrangement of equipment according to claim 4, wherein the therapeutic device is a remote-controlled medicine-dosing device.

18. The arrangement of equipment according to claim 17 wherein the therapeutic device is an implanted insulin pump.

19. The arrangement of equipment according to claim 1, wherein the interface of the function-measurement device comprises a first mobile wireless terminal with data input.

20. The arrangement of equipment according to claim 3, wherein the second display unit with integrated interface comprises a first mobile wireless terminal.

21. The arrangement of equipment according to claim 1, wherein the interface of the first display unit comprises a second mobile wireless terminal with data output.

22. The arrangement of equipment according to claim 21, wherein the second mobile wireless terminal is a hand-held PC with an integrated mobile wireless element.

23. The arrangement of equipment according to claim 1, wherein the outward channel transmits data per SMS or EMS or WAP-push with automatic transmission activation by way of the function-measurement device.

24. The arrangement of equipment according to claim 1, wherein the return channel transmits data per SMS or EMS or WAP-push with manual transmission control.

25. The arrangement of equipment according to claim 1, wherein the outward and return channels comprise permanent packet-oriented data connections according to GPRS or UMTS.

26. The arrangement of equipment according to claim 1, further comprising:
a data bank associated with the first display unit, for the storage of transmitted mammal-related measurement data or function characterizations; and
a data-bank management system for the management of the data bank, including output to authorized users.

27. The arrangement of equipment according to claim 24, wherein at least one of the data bank and the data-bank management system is connected by way of an internet portal to one of a public data and a telecommunication network used to transmit one of the measured values and the function characterizations.

28. The arrangement of equipment according to claim 27, further comprising:
means for testing the authorization of a potential user, associated with the internet portal; and
access-control means connected to the authorization testing means for allowing access to the data bank in response to a positive result of the authorization test.

29. The arrangement of equipment according to claim 1, wherein the bi-directional public telecommunication network connection comprises a transmission section in an IP network and at the boundaries of this transmission section there are provided gateways to create a connection to the public telecommunication network and to convert the transmission format.

30. The arrangement of equipment according to claim 29, wherein the gateways convert between at least one of SMS and e-mail, SMS and fax, and e-mail and fax.

31. An arrangement of equipment for the remote monitoring of bodily functions of a mammal under medical care comprising:
a function-measurement device to measure a physiological quantity relevant to bodily function;
an automatic monitoring and control unit disposed remotely from said function-measurement device for evaluating at least one of the measured values of the physiological quantity and a function characterization derived therefrom, and to generate control commands in response to the result of the evaluation for the function-measurement device; and
a bi-directional public telecommunication-network connection with an outward channel between the function-measurement device and the automatic monitoring and control unit and a return channel between said automatic monitoring and control unit and the function-measurement device, wherein for the connected device there is provided an interface adapted to a transmission protocol of the telecommunication network.

32. The arrangement of equipment according to claim 31, further comprising:
a video camera disposed at the mammal; and
a video receiving and display device disposed at a distance from the video camera and connected thereto by the outward channel of the public telecommunication-network connection, to receive and display the pictures of the mammal recorded by the video camera.

33. The arrangement of equipment according to claim 31, further comprising a first display unit disposed at the mammal to display warning signals or information generated by the monitoring and control unit.

34. The arrangement of equipment according to claim 31, further comprising at least one therapeutic device disposed at the mammal to provide a therapy in response to a control command generated by the monitoring and control unit.

35. The arrangement of equipment according to claim 31, further comprising:
a wireless short-range connection between the function-measurement device and an additional device disposed at the mammal; and
an interface of the telecommunication network associated with the wireless short-range connection, according to one of the Bluetooth, wireless LAN, and DECT standards.

36. The arrangement of equipment according to claim 35, wherein said additional device is one of a video camera, a therapeutic device, and a first display unit.

37. The arrangement of equipment according to claim 31, wherein the function-measurement device is one of a pulse meter to detect the pulse rate and a cardiac-rhythm detector.

38. The arrangement of equipment according to claim 31, wherein the function-measurement device is a blood pressure meter.

39. The arrangement of equipment according to claim 31, wherein the function-measurement device is an oximeter device to detect the blood oxygen content.

40. The arrangement of equipment according to claim 31, wherein the function-measurement device is a glucose-measurement device to detect the blood-sugar content.

41. The arrangement of equipment according to claim 31, wherein the function-measurement device is a device to detect one of a respiratory rhythm and pulmonary performance.

42. The arrangement of equipment according to claim 31, wherein the function-measurement device is an EEG-recording device to detect the brain currents.

43. The arrangement of equipment according to claim 31, wherein the function-measurement device comprises an evaluation device to obtain a function characterization from the collected data.

44. The arrangement of equipment according to claim 34, wherein the therapeutic device is a remote-controlled, implanted cardiac pacemaker.

45. The arrangement of equipment according to claim 34, wherein the therapeutic device is a remote-controlled, implanted defibrillator.

46. The arrangement of equipment according to claim 34, wherein the therapeutic device is a remote-controlled breathing aid with an oxygen or aerosol generator.

47. The arrangement of equipment according to claim 34, wherein the therapeutic device is a remote-controlled medicine-dosing device.

48. The arrangement of equipment according to claim 47, wherein the remote-controlled medicine-dosing device is an implanted insulin pump.

49. The arrangement of equipment according to claim 31, wherein the interface of the function-measurement device comprises a first mobile wireless terminal with data input.

50. The arrangement of equipment according to claim 33, wherein the first display unit comprises an integrated interface and is a first mobile wireless terminal.

51. The arrangement of equipment according to claim 50, wherein the interface of the first display unit comprises a second mobile wireless terminal with data output.

52. The arrangement of equipment according to claim 51, wherein the second mobile wireless terminal comprises a hand-held PC with an integrated mobile wireless element.

53. The arrangement of equipment according to claim 34, wherein an interface of the therapeutic device comprises a second mobile wireless terminal with data output.

54. The arrangement of equipment according to claim 31, wherein the outward channel transmits data per one of SMS, EMS, and WAP-push with automatic transmission activation by way of the function-measurement device.

55. The arrangement of equipment according to claim 31, wherein the return channel transmits data per one of SMS, EMS, and WAP-push with one of manual transmission control and transmission control by means of the monitoring and control unit in response to the result of the evaluation.

56. The arrangement of equipment according to claim 31, wherein the outward and return channels comprise permanent packet-oriented data connections according to one of GPRS and UMTS.

57. The arrangement of equipment according to claim 31, further comprising:
a data bank associated with at least one of the first display unit and the monitoring and control unit, for the storage of transmitted mammal-related measurement data and function characterizations; and
a data-bank management system for the management of the data bank, including output to authorized users.

58. The arrangement of equipment according to claim 57, wherein at least one of the data bank and the data-bank management system is connected by way of an internet portal to at least one of a public data and a telecommunication network used to transmit the measured values and the function characterizations.

59. The arrangement of equipment according to claim 58, further comprising:
means for testing the authorization of a potential user, associated with the internet portal; and
access-control means connected to the authorization testing means to allow access to the data bank in response to a positive result of the authorization test.

60. The arrangement of equipment according to claim 31, wherein the bi-directional public telecommunication-network connection comprises a transmission section in an IP network and at the boundaries of the transmission section there are provided gateways to create a connection to the public telecommunication network and to convert the transmission format.

61. The arrangement of equipment according to claim 60, wherein the gateways are convert between at least one of SMS and e-mail, SMS and fax, and e-mail and fax.
